(19) United States
(12) Patent Application Publication
(10) Pub. No.: US 2017/0333119 A1
(43) Pub. Date: Nov. 23, 2017

(54) SURGICAL DEVICE HAVING AXIALLY RECIPROCATING ELECTRODE ASSEMBLY AND METHODS FOR TREATING PROSTATE

(71) Applicant: Corinth MedTech, Inc., Cupertino, CA (US)

(72) Inventor: Csaba Truckai, Saratoga, CA (US)

(21) Appl. No.: 15/603,220

(22) Filed: May 23, 2017

Related U.S. Application Data
(60) Provisional application No. 62/340,945, filed on May 24, 2016.
(60) Provisional application No. 62/340,446, filed on May 23, 2016.

(51) Int. Cl. A61B 18/14; A61B 18/12; A61B 18/00

(52) U.S. Cl. CPC A61B 18/1482; A61B 18/1206; A61B 2018/0091; A61B 2018/00607; A61B 2018/00601; A61B 2018/00547; A61B 2018/00184; A61B 2018/00166; A61B 2018/1475; A61B 2018/00589

(57) ABSTRACT

A tissue resecting device includes a handle, a shaft assembly movably attached to the handle, a housing secured to a distal end of the shaft, and an electrode. The electrode is disposed in the housing to move across a window, and at least one motor in the handle both reciprocates the shaft assembly relative to the handle and drives the electrode across the window.

### CROSS-REFERENCE TO RELATED APPLICATIONS

This application claims the benefits of provisional application No. 62/340,945 (Attorney Docket No. 42005-709.101), filed on May 24, 2016, and of provisional application No. 62/340,446 (Attorney Docket No. 42005-708.101), filed on May 23, 2016, the full disclosures of which are incorporated herein by reference.

### BACKGROUND OF THE INVENTION

### 1. Field of the Invention

The present invention relates to devices and methods for resecting and removing tissue from an interior of a patient's body, for example in a transurethral resection of prostate tissue to treat benign prostatic hyperplasia.

Electrosurgical cutting devices often comprise a shaft or sleeve having a tissue extraction lumen with one or more radio frequency (RF) cutting blades arranged to resect tissue which may then be drawn into the extraction lumen, often via vacuum assistance through a cutting window. Most such electrosurgical tissue cutting devices rely on manually engaging the cutting window against the target tissue to be resected. While such manual engagement is often sufficient, in other cases, such as in laparoscopic procedures having limited access and field of view, the target tissue can be difficult to visualize prior to resection and, in particular, it can be difficult to assure that the optimum target site has been engaged by the cutting window. For these reasons, it would be desirable to provide improved electrosurgical cutting tools having improved visibility and ability engage and immobilize tissue prior to cutting and to extract the tissue from tools after cutting.

For resection of remote tissue sites, such as the prostate, it is usually desirable to introduce the surgical cutter through a tubular introducer device. Which such tubular introducers can be advanced “blind,” i.e. without direct optical visualization, it is frequently desirable to prove such direct visualization. For example, it would be desirable to use an endoscope to observe the urethra while transurethrally advancing an introducer sheath for subsequent resection of the prostrate. Once the introducer sheath is in place and the surgical cutter has been introduced, however, it will still be necessary to move a cutter element on the surgical cutter to resect the tissue. Heretofore, this has typically been accomplished by manually reciprocating a cutter assembly on the tissue resecting apparatus. Manual resection, while generally effective, can be difficult to control and, in particular, can be difficult to coordinate with other aspects of the resection procedure, such as applying RF power, applying a vacuum to aspirate tissue fragments and debris, and the like.

For these reasons, it would be desirable to provide improved apparatus, systems and methods for resecting tissue in prostatectomies and other procedures. It would be particularly desirable to provide apparatus, systems and methods which provide improved control of tissue resection including but not limited to enhanced coordination of cutter movement control, cutting power control, vacuum aspiration control, and the like. At least some of these objectives will be met by the inventions described below.

### 2. Description of the Background Art

Commonly owned patents and published applications disclosing related subject matter include U.S. Pat. No. 8,221,404; U.S. Pat. No. 7,744,595; U.S. Pat. Publs. US2017/0105748; 2014/0336643; U.S. Pat. Publ. 2010/0305565; U.S. Pat. Publ. 2007/0213704; U.S. Pat. Publ. 2009/0270849; and U.S. Pat. Publ. 2013/0090642.

### BRIEF DESCRIPTION OF DRAWINGS

FIG. 1 is a view of a tissue resecting device and a block diagram of systems and operating components corresponding to the invention.

FIG. 2 is a perspective view of the working end of the resecting device of FIG. 1 showing an asymmetric ceramic housing and moving electrode that is adapted to sweep across a tissue-receiving window.

FIG. 3 is another perspective view of the working end of FIG. 2 from a different angle.

FIG. 4A is a schematic view of the working end of FIGS. 2-3 interfacing with tissue targeted for resection under endoscopic vision.

FIG. 4B is a schematic view of a working end of a prior art tubular cutting device used in a hypothetical resection procedure.

FIG. 5 is another schematic view of the working end of FIGS. 2-3 being used to resect targeted tissue to a significant depth from the organ surface.

FIG. 6 is a perspective view of a distal dielectric housing of a working end similar to that of FIGS. 2-3 showing window sides with ledges for receiving the electrode at the ends of its movement in a sweeping arc.

FIG. 7A is a perspective view of a distal ceramic housing of a working end similar to that of FIG. 6 with the distal tip of the moveable electrode adapted to move in a constraining slot or channel.

FIG. 7B is a perspective view of an alternative ceramic housing similar to that of FIG. 7A with the distal tip of the moveable electrode adapted to pivot or rotate in a bore or pivot.

FIG. 8 is a perspective view of a tissue resecting device that includes a motor drive for moving the shaft assembly and working end in a reciprocating mode relative to the handle.

FIG. 9A is a perspective view of the working end of the device of FIG. 8 showing an endoscope carried by the shaft assembly and the endoscope field of view.

FIG. 9B is a perspective view of the working end of the device of FIG. 9A from another angle.

FIG. 10A is a side view of the tissue resecting device of FIG. 8 with the reciprocating shaft assembly and working end at the distal end of an extending stroke relative to the handle.

FIG. 10B is a side view of the tissue resecting device of FIG. 10A with the reciprocating shaft assembly and working end at the proximal end of a retracting stroke relative to the handle.

FIG. 11 is a sequential view of the tissue resecting device of FIGS. 10A-10B showing retracting and extending strokes and a method of activating and de-activating the negative pressure source and the delivery of RF current to the electrode in different portions of the retracting and extending strokes.

FIG. 12A is a sectional schematic view of the working end with the moving electrode resecting tissue.

FIG. 12B is a sectional schematic view of the working end similar to that of FIG. 12A with a stationary electrode coagulating tissue.

### SUMMARY OF THE INVENTION

The present invention provides apparatus, systems, and methods for performing electrosurgical resections in minimally invasive procedures. While the apparatus, systems, and methods are particularly suitable for performing transurethral resection of the prostate (often referred to as TURP), they will also find use in a variety of other laparoscopic and other endoscopic and endosurgical procedures. The apparatus comprises motor-driven cutters, where the motors are configured to drive both a shaft of the cutter and a cutter electrode, either independently, contemporaneously, or selectively independently and contemporaneously. The systems comprise the cutters together with a digital or other controller configured to coordinate movements of the shaft, electrodes, and other external components such as a radiofrequency power supply (e.g. by selecting a cutting or a coagulation waveform, power, timing, etc.), a negative pressure source, and the like. The methods of the present invention comprise using the apparatus and systems as just described for prostatectomies and other tissue resection procedures.

In a first aspect, the present invention provides a tissue resecting device comprising a shaft assembly movably attached to a handle and having a longitudinal axis. A housing is secured to a distal end of the shaft and has a window configured to be fluidly coupled to a negative pressure source. An electrode is disposed in the housing and configured to move relative to the window, and at least one motor in the handle is adapted to both (1) move the shaft assembly in an axial stroke relative to the handle and (2) move the electrode across the window.

In specific embodiments and examples of the tissue resecting device, the at least one motor is adapted to move the shaft assembly and the electrode contemporaneously, i.e. at the same time. In other specific embodiments and examples, the at least one motor is adapted to selectively move either the shaft assembly or the electrode individually. In many embodiments, the at least one motor will be adapted to move the shaft assembly and electrode both contemporaneously and individually at different times during a procedure. In still additional specific examples, the motor will be adapted to move the electrode at a fixed speed or rate relative to the window, e.g. at a rate greater than 1 cycle per second (CPS), often greater than 5 CPS. The motor may be still further adapted to reciprocate the shaft assembly at a rate greater than once every two seconds, frequently at a rate greater than once every second.

The shaft and/or the electrode may be operated manually and/or automatically. That is, the user may be able to manually initiate the at least one motor to move the electrode in the housing relative to the window and/or to manually activate the at least one motor to reciprocate the shaft in an axial stroke relative to the handle. Even when being operated manually, the tissue resecting device will usually be operated through an interface (typically including a radiofrequency (RF) power supply) which may provide for specific operational parameters, often fixed or manually adjustable parameters, such as stroke times, power levels, RF waveforms, and the like, without having feedback control capability.

Often, however, the tissue resecting device will be provided as part of a tissue resecting system which further comprises a controller which is configured to operate not only the motor, but usually also a RF power source which is coupled to the electrode and also a negative pressure source which may be coupled to the window in the housing. The controller may be further configured or adapted to automatically or manually control at least one motor to stop movement of the electrode in a selected position relative to the window. Alternatively or additionally, the controller may be adapted to stop the electrode in the center of the window. Alternatively or additionally, the controller may be adapted to stop the electrode at an end of the window.

The controller may be adapted in a variety of other different control protocols. For example, the controller may be adapted to control the at least one motor to provide a single movement cycle of the electrode back and forth across the window. That is, the user may be able to cause the controller to initiate only a single pass of the electrode over the window in order to achieve a controlled cutting of tissue. In other instances, the controller may be adapted to control the at least one motor to stop axial movement of the shaft in a selected axial position. The controller may be further adapted to control the at least one motor to provide a single movement of the shaft in retracting and/or extending stroke. Additionally, the controller will usually be configured to control and coordinate the delivery of negative pressure from the negative pressure source to the housing window and to actuate the at least one motor, usually contemporaneously.

In still other aspects of the control systems of the present invention, the controller may be configured to modulate the negative pressure source in response to movement of the shaft assembly. That is, the negative pressure may be applied only, for example, when the shaft is extend and/or may be deactivated only when the shaft is retracted.

In still further aspects of the systems of the present invention, the controller may be configured to modulate the negative pressure source in response to movement of the electrode relative to the window. For example, the controller may be configured to active or deactivate the RF source in response to movement of the electrode relative to the window. Still additionally, the controller may be configured to activate or deactivate the RF source to deliver a cutting current waveform or a coagulation waveform to the electrode.

In a second aspect, a tissue resecting system comprises a handle, an elongate shaft, an electrode, and a controller. The elongate shaft is reciprocatably connected to the handle and extends along a longitudinal axis to a working end. The working end is movable in a stroke between a first axial position and a second axial position relative to the handle. The electrode is disposed at the working end of the shaft and is configured to be coupled to an RF source. An aspiration channel is formed in the elongate shaft and communicates with a window in the working end of the shaft and is configured to be coupled to a negative pressure source. The controller is operatively connected to the RF source and the negative pressure source and is configured to modulate energy delivery from the RF source to the electrode and to modulate negative pressure to the aspiration channel where modulations of both pressure and energy are in response to an axial position of the working end in said stroke.

In a third aspect, a method of the present invention for resecting tissue comprises providing an elongate shaft assembly. The elongate shaft assembly includes an electrode proximate a window in a housing. A motor reciprocates the shaft assembly in a retracting stroke and an extending stroke relative to a handle. The handle is manipulated to position the electrode against a targeted tissue site, and a negative pressure source may be activated to communicate with the window in the working end to draw tissue to or through the window. The RF source is then activated to deliver RF current to the electrode, and the motor is controlled to reciprocate the shaft assembly in a retracting stroke to resect tissue. Optionally, the motor may further laterally reciprocate or otherwise drive the electrode in a lateral stroke across the window to effect tissue resection.

In specific embodiments and examples, the steps of activating the negative pressure source, activating the RF source, and controlling the motor are performed by a digital or other controller. The methods may further comprise deactivating the negative pressure source at the proximal end of the retracting stroke. The methods may alternatively or additionally comprise deactivating the RF source at the proximal end of the retracting stroke. The methods may still further alternatively or additionally comprise commencing the extending stroke with the negative pressure source deactivated, commencing the extending stroke with the RF source deactivated, activating the negative pressure source during a portion of the extending stroke, and/or activating the negative pressure during a terminal portion of the extending stroke.

In particular aspects of the present invention as described in detail below, the devices, systems and methods are particularly configured for treating the prostate, optionally under endoscopic visualization. For example, the systems may comprise a RF source configured to deliver RF current alternatively in a cutting waveform and a coagulation waveform to the electrode, a motor configured to move the electrode, and a controller configured to operate the motor and RF source in a first mode delivering a cutting waveform while activating the motor to move the electrode and in a second mode delivering a coagulation waveform after de-activating the motor to stop the electrode in a selected stationary position. Such methods for treating the prostate may comprise providing a treatment device with a shaft extending along a longitudinal axis to a distal portion having a window communicating with an aspiration source and a motor driven electrode adapted to move relative to the window. The window is engaged against targeted prostate tissue, and the RF source is operated in a first mode with a cutting waveform delivered to the electrode while activating the motor to move the electrode to resect tissue and thereafter operated in a second mode with a coagulation waveform delivered to the electrode after de-activating the motor to stop the electrode in a selected stationary position to coagulate tissue.

### DETAILED DESCRIPTION OF THE INVENTION

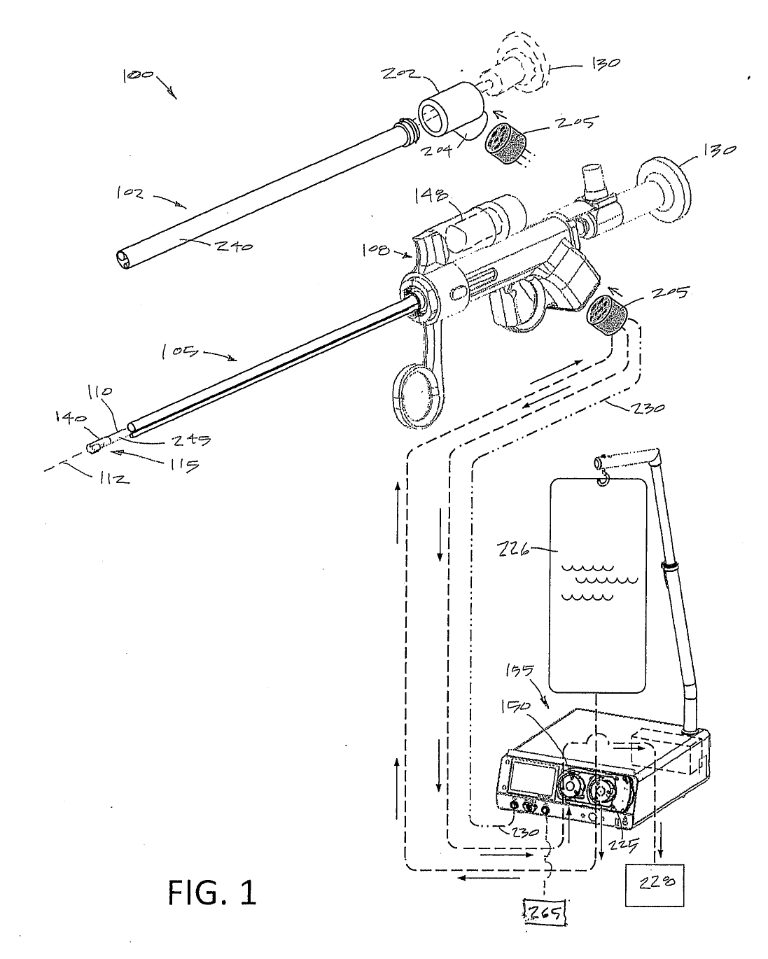

FIGS. 1 illustrates an electrosurgical tissue resecting system 100 for use in urological procedures to resect tissue that includes an introducer sleeve or sheath 102 and a hand-held single-use tissue resecting device or probe 105. The resecting device 105 has a handle portion 108 that is coupled to an elongated shaft or extension portion 110 that has an outer diameter ranging from about 2 mm to 7 mm, and in one variation is 5 mm in diameter. The shaft 110 extends about longitudinal axis 112 to a working end 115 that is radially asymmetric relative the shaft 110 and its axis 112 as further described below. In one variation, the device is adapted for performing a TURP procedure (transurethral resection of prostate) or a bladder tumor resection procedure and thus the shaft portion 110 extends about axis 112 with a length suitable for introducing in a transurethral approach to reach the targeted prostate tissue or bladder tissue.

As will be described below and shown in FIG. 1, the resecting device 105 is adapted for introduction through the introducer sleeve 102. Such an introducer sleeve 102 is adapted to receive a commercially available endoscope 130 as can be understood from FIG. 1.

Referring to FIGS. 1-3, in general, it can be seen the resecting device 105 has an elongated shaft 110 that extends to a distal shaft portion 132 that is coupled to an offset resecting housing 140 that has an offset tissue-receiving window 144. A moveable electrode 145 is adapted to be driven by a motor drive unit 148 in handle 108 (see FIG. 1) so that the longitudinal portion 149 of the electrode 145 sweeps across the window 144 from side to side to electrosurgically resect tissue that is captured in the window 144. The targeted tissue can be suctioned into and captured in window 144 by means of a negative pressure source or outflow pump 150 in controller 155 that communicates with a tissue extraction channel 158 extending through the device 105 and terminating in the window 144.

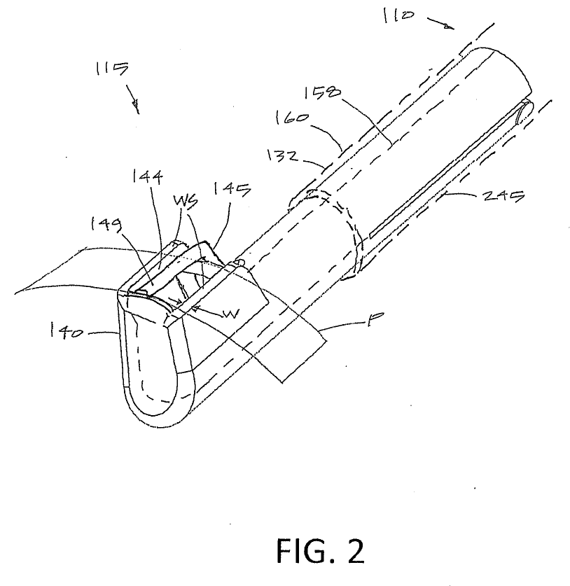

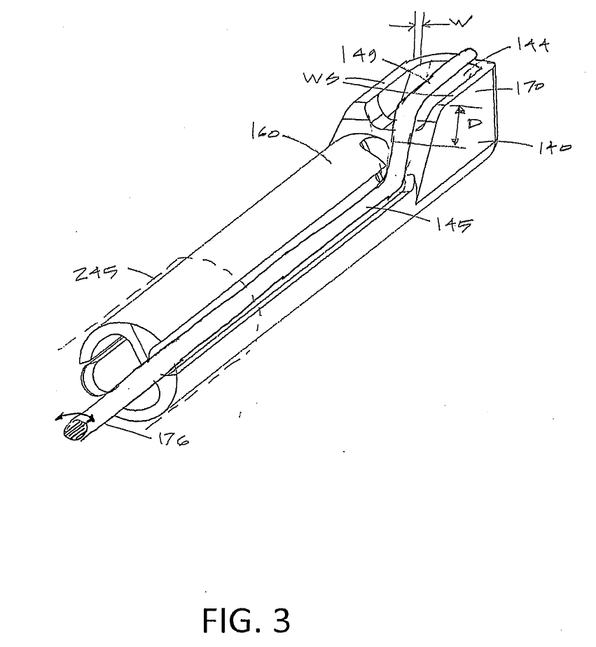

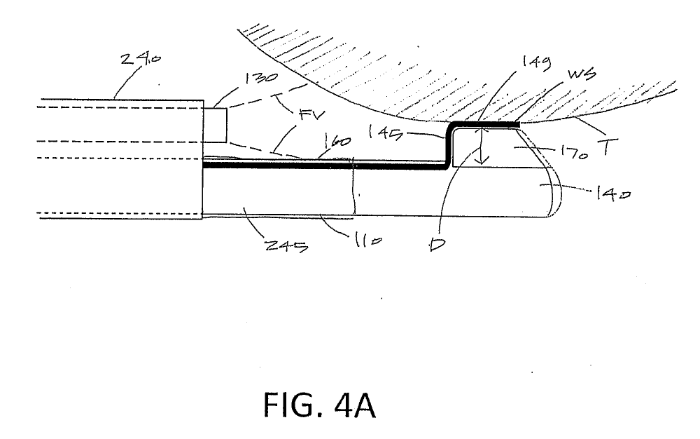

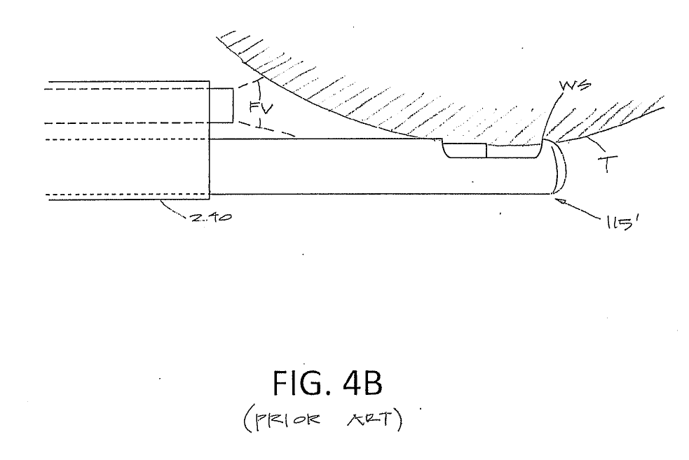

More in particular, referring to FIGS. 2 and 3, the configuration of the offset housing 140 is adapted to perform multiple functions. First, the offset housing 140 positions the window surface WS (within curved plane P indicated in FIG. 2) outwardly from the outer surface 160 of shaft 110 which then allows the window surface WS to be fully visible through a endoscope 130 or other viewing means that would be introduced parallel to the device shaft 110 (see FIG. 4A). For example, FIG. 4A is a schematic view of the working end 115 with working surface WS in contact with targeted tissue T. As can be seen in FIG. 4A, the endoscope 130 is positioned with the field of view FV directly aligned with the working surface WS thus allowing optimal viewing of the tissue resection process.

In contrast, FIG. 4B shows a working end 115′ of a conventional dual sleeve tubular cutter having a window surface WS′ which when pressed against an organ prevents endoscopic vision of the interface between the tubular cutting edge and the tissue T during a resection procedure.

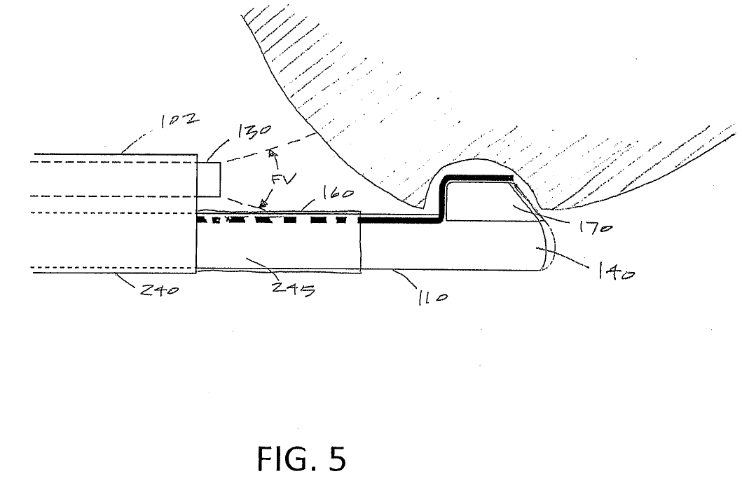

Second, the offset housing 140 is adapted for resecting tissue to a greater depth in a localized region of an organ, rather than resecting surface tissues over a broad area. More in particular as shown in FIG. 5, the offset portion 170 of housing 140 can be pushed into tissue perpendicular to axis 112 of the probe shaft 110. Thus, as shown in FIG. 5, the offset housing 140 can be used to resect tissue deep into in a localized region that would not be possible with a resecting device having the configuration shown in FIG. 4B.

FIGS. 2 and 3 illustrate the asymmetric or offset dielectric housing 140 that can comprise a ceramic material such as zirconium oxide, aluminum oxide or similar materials as is known in the art. In FIGS. 2-3, it can be seen that window surface WS is offset from the shaft surface 160 by a predetermined dimension D which can be from 2 mm to 8 mm and in one embodiment comprises a 5 mm offset.

As can be further be seen in FIGS. 2-3, the width W of the window surface WS around at least portions of the perimeter of the window 144 is a limited dimension, for example less than 3 mm, or less than 2 mm or less than 1 mm. which allows the offset portion 170 of housing 140 to be pushed into tissue perpendicular to the device axis 112 as the electrode 145 sweeps across the window 144.

Referring to FIGS. 2-3, one variation of resecting device 105 has an electrode 145 that can be tungsten or stainless steel wire that with electrode portion 149 adapted to sweep across the window 144 at any suitable rate, for example from 1 cycle per second (CPS) to 50 CPS or more. In FIG. 3, it can be understood that the electrode 145 has an elongated proximal shaft portion 176 that extends into handle 108 of the device (FIG. 1). The proximal end of electrode 145 is operatively coupled to a motor drive unit 148 and a suitable mechanism or controller is provided to rotate the elongated electrode shaft portion 176 in an arc to resect tissue.

As can be understood from FIGS. 2-3, the electrode portion 149 moves back and forth akin to a windshield wiper across window 144 in the offset housing 140. A number of mechanisms can be used to effectuate the desired movements of the electrode, or the motor drive 148 simply can be controlled by software to move in intermittent clockwise and counter-clockwise directions. In one variation, the elongated proximal portion 176 of the electrode 145 will twist over its length and thus the motor drive 148 can be adapted to rotate the electrode shaft in an arc with radial angle which is greater than the window's comparable radial angle or arc. Thus, the electrode portion 149 can be expected to move back and forth entirely across the window even when meeting some tissue resistance by compensating for some twisting that is allowed in the proximal electrode shaft portion 176. In one variation, the motor drive unit can be adapted to over-rotate the electrode shaft portion 176 at its proximal end by a selected amount which can be from 10° radial motion to 90° radial motion to compensate for twisting of the electrode shaft portion to insure that electrode portion 149 sweeps entirely across the surface of window 144.

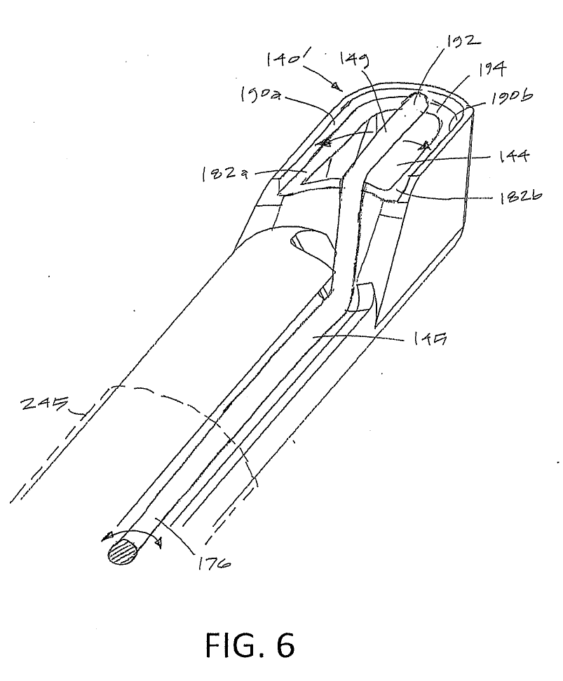

In general, the window 144 in housing 140 can be configured to have a radial arc relative to the electrode shaft 176 ranging between 30° and 180°. In one variation of housing 140′ shown in FIG. 6, it can be seen that the electrode portion 149 has a range of motion that extends across the radial dimension of the window 144 to ensure that any tissue captured in the window is resected as the electrode portion 149 passes the window edges 182a and 182b to function like a shear or in a scissor-like manner. The electrode portion 149 moves over ledges 186a and 186b on either side of the housing 140′ and can bump into surfaces 190a and 190b. By bumping into the surfaces 190a and 190b, any over rotation in the electrode shaft 176 to accommodate twisting as described above can limit the rotation of the electrode portion in the housing 140′. Further, in FIG. 6, it can be seen that the distal tip 192 of electrode portion 149 extends distally beyond window 144 and onto distal ledge 194 in the housing 140′ to ensure tissue is resected by the electrode in the distal window region.

Now turning back to FIG. 1, it can be understood that the resecting device 105 and endoscope 130 can be used with introducer sleeve assembly or sheath 102. As shown in FIG. 1, the introducer assembly 102 has a proximal handle body 202 with a connector 204 that is adapted to couple to connector member 205. The connector 205 is adapted to couple a conduit 206 to controller 155 and provide within a single cable the following: (i) a first lumen communicating with the fluid outflow pump 150, (ii) a second lumen communicating with a fluid inflow pump 225, and (iii) a third lumen communicating with a pressure sensor positioned in the controller 155 or in or near the connector 205. As can be seen in FIG. 1, the introducer sleeve 102 can also accommodate an endoscope 130. Thus, the introducer sleeve 120 can be assembled with the endoscope 130 (and without the resection device 105) and coupled by connector 205 to the controller 155 to provide an inflow of irrigation fluid from fluid source 226, and outflow of irrigation fluid to collection reservoir 228 together with pressure sensing to allow the assembly to be used in a diagnostic procedure prior to a tissue resection procedure. In other words, the introducer sleeve 102 can function as a ‘continuous flow’ optical introducer for use in trans-urethral access to a targeted sire in the prostate or bladder.

After the introducer sleeve assembly 102 is used for an initial diagnostic procedure, the endoscope 130 can be removed from the assembly 102 and connector 205 can be disconnected from handle body 205. Thereafter, the sleeve portion 240 (see FIG. 1) of introducer assembly 102 can be detached from proximal handle body 204 with the sleeve portion 240 remaining in the patient. Next, the endoscope 130 and connector 205 can be assembled with the resecting device 105 and the physician can insert the resecting device 105 through the sleeve portion 240 remaining in the patient to access the targeted site. The resecting device 105 and sleeve portion 204 in combination then provide lumens as described above for fluid inflows, fluid outflows and direct pressure sensing through lumens in connector 205.

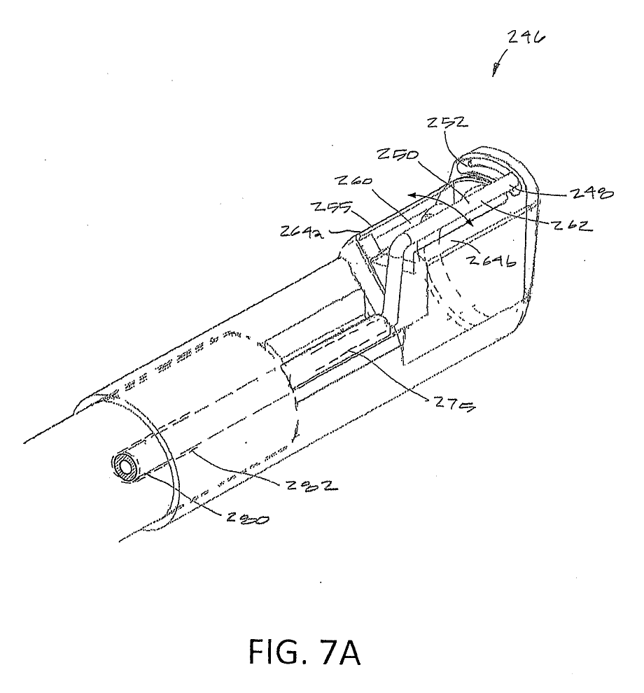

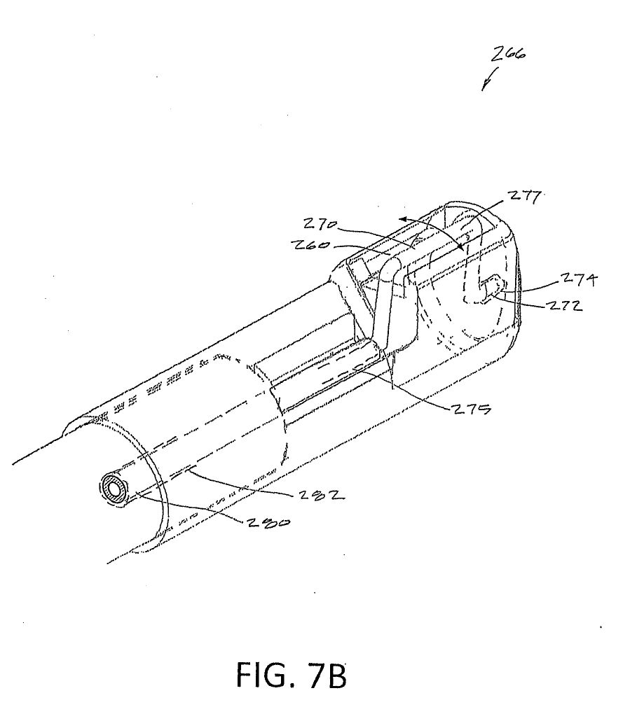

Now turning to FIG. 7A, a perspective view of a distal ceramic housing of a working end 246 similar to that of FIG. 6 is shown. In this variation, the distal tip 248 of the moveable electrode 250 is configured to be constrained within a constraining slot or channel 252. In other words, the distal electrode tip 248 is not free-floating as in the variation of FIG. 6. It has been found that an electrode with a free-floating distal tip can be caught by tissue and be lifted away from the ceramic housing 255. Thus, in this variation the distal electrode tip 248 is constrained and cannot be tangled with tissue or lifted away from the ceramic housing and window 260. The variation of FIG. 7A illustrates an arcuate slot or channel 252 that limits the movement of the electrode 250. In all other respects, the working end functions as described previously. Further, the distal electrode portion 262 and channel 252 can be configured to allow the electrode to pass over the edges 264a and 264b of the window 260 as described above.

FIG. 7B shows another variation of working end 266 in which the electrode 270 has a distal tip 272 that is constrained in a pivot or bore indicated at 274. In this variation, it can be seen that the electrode 270 has a U-shape with the distal tip 272 aligned with the electrode shaft portion 275 to allow the active electrode portion 277 to move from side to side relative to window 260 as described previously.

In another aspect of the invention shown in FIGS. 7A-7B, the electrode shaft portion 275 comprises a tubular member 280 which can comprise a metal hypotube, such as stainless steel or a similar material. In a previous variation as shown in FIG. 6, the electrode shaft portion comprised a wire element which could potentially twist to an unwanted degree when the electrode engaged dense tissue, for example. In this variation, it has been found that a metal hypotube with a suitable wall thickness can resist twisting when the electrode is being moved and engaging dense tissue. In one variation, the wall thickness of the tubular member 280 can be at these 0.005″ or at least 0.010″.

In general, a tissue resecting device corresponding to the invention comprises an elongated member extending along a longitudinal axis to a distal portion having a window communicating with an aspiration source, an electrode having an electrode shaft with a central axis extending within the elongated member to an electrode working end wherein a portion of the electrode working end is offset from said central axis, and a motor configured to rotate the electrode shaft to cause the electrode working end to move relative to the window wherein the electrode shaft comprises a tubular member adapted to resist twisting of said shaft during motor driven movement thereof. Further, the tubular member can comprise a metal tube with an insulative outer surface layer 282. The tissue tubular member can be a stainless steel tube with the insulative outer surface layer comprising a heat shrink polymer.

In one variation, the electrode's working end has a profile that is substantially smaller than the area of the window to thereby permit fluid aspiration around the electrode working end at all times through the window as the electrode is moving relative to the window. This allows the negative pressure source to draw the tissue into the window interface, and maintains the tissue in the interface as the electrode cuts and extracts the resected tissue. In one variation, the electrode working end is motor driven and moves at a rate of equal to or greater than 1 CPS relative to the window, or equal to or greater than 10 CPS relative to the window. As described previously, the electrode working end can be offset radially outward from the shaft assembly by at least 2 mm or by at least 4 mm.

In another aspect of the invention, the tissue resecting device comprises an elongated member extending to a distal housing having a tissue-receiving window, a moveable electrode configured to move across the window, and a motor configured to move the electrode wherein a distal tip of the electrode moves in a constraining channel in the housing. In another variation, the tissue resecting device comprises an elongated member extending to a distal housing having a tissue-receiving window, a moveable electrode configured to move across the window; and a motor configured to move the electrode wherein a distal end of the electrode is non-free floating or pivots in a pivot channel.

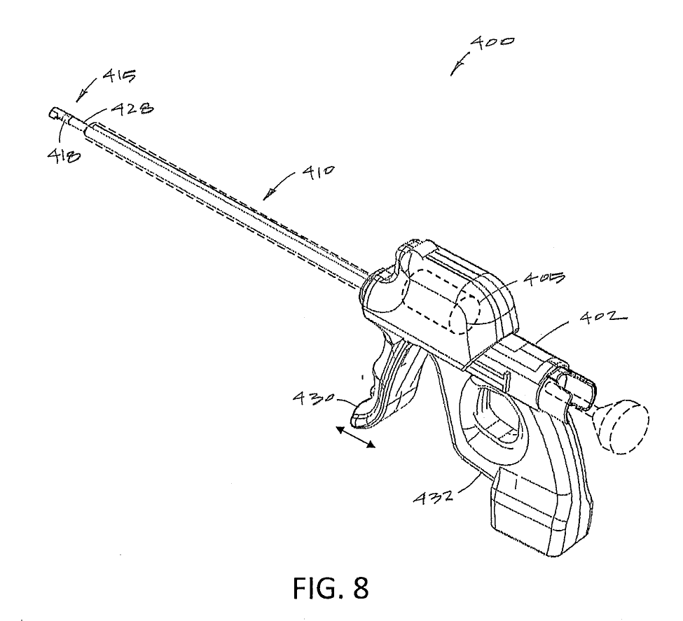

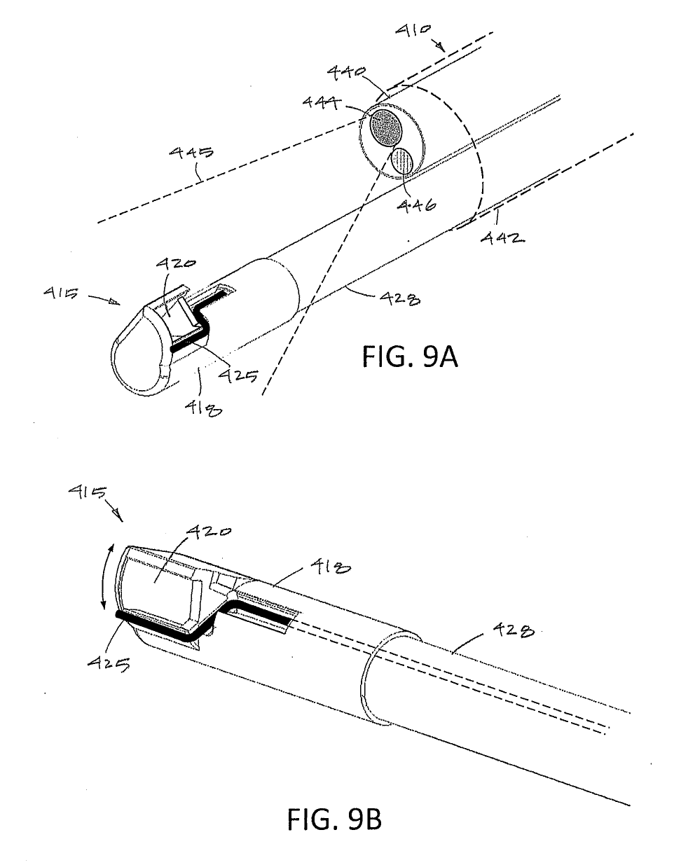

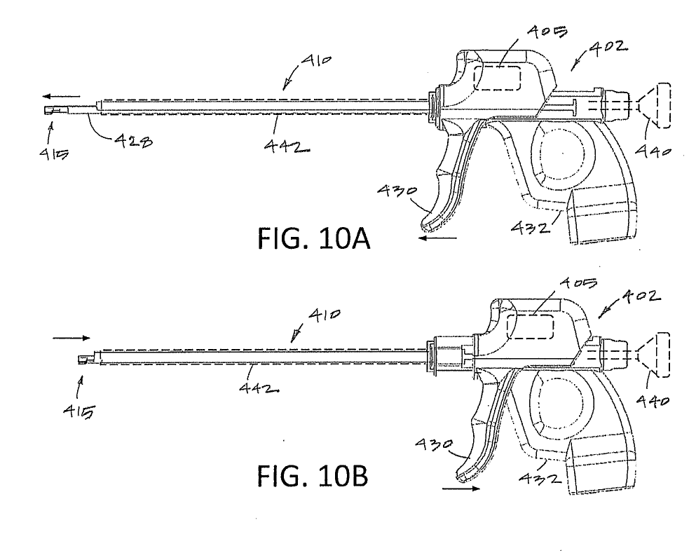

FIG. 8 is a perspective view of a tissue resecting device 400 that includes a handle 402 carrying a motor drive 405 and a shaft assembly 410 extending from the handle to a working end 415, for example comprising a ceramic or other housing 418 (FIGS. 9A and 9B) having a tissue-receiving window 420 and a motor-driven electrode 425 that is adapted to move across the window 420 as described previously. The working end 415 is coupled to sleeve 428 which is adapted for manual or motor-driven reciprocation within shaft assembly 410. More in particular, this variation of device 400 provides the motor drive 405 for moving the electrode 425 in the working end 415 which is similar to that of FIG. 7A. Further, in this embodiment, the device 400 can optionally utilize the motor drive to reciprocate the working end 415 relative to the shaft assembly 410 contemporaneously or alternatingly with the movement of the electrode 425 relative to the window 420 as described previously. Alternatively, the device 400 carries a first motor for moving the electrode 425 relative to the window 420 in the housing 418 and a second motor (not illustrated) for reciprocating the working end 415. In another variation, the single motor 405 can be adapted to perform both the electrode movement and the working end reciprocation. As can be seen in FIGS. 10A-10B, the handle 402 allows for manual retraction and extension of the working end 415 within the shaft assembly 410 by movement of an actuator grip 430 relative to stationary grip portion 432 of handle 402.

FIG. 9A is a perspective view of the working end 415 of the device 400 of FIG. 8 showing an endoscope 440 carried with an outer sleeve 442 of the shaft assembly 410. The working end 415 carried by sleeve 428 is similar to that of FIGS. 2, 3, and 6 described previously, but could have any of the constructions described previously. The endoscope 440 has optics 444 which provide a field of view 445 which can encompass the working end 415 on the elongated member 428. A light emitter 446 is shown in the distal end of the endoscope 440. FIG. 9B is a perspective view of the working end of the device of FIG. 9A from another angle.

FIGS. 10A and 10B are side views of the tissue resecting device 400 of FIG. 8 illustrating reciprocation of the sleeve 428 and working end 415 within shaft assembly 410 and relative the stationary grip portion 432 of handle 402. FIG. 10A shows the sleeve 428 and working end 415 at a distal end of an extending stroke relative to the shaft assembly 410 and handle 402, and FIG. 10B shows at the working end 415 and sleeve 428 at a proximal end of a retracting stroke relative to the handle. In this variation, the working end 415 and sleeve 428 are adapted to reciprocate while the endoscope 440 remains stationary in the handle 402. In alternative embodiments (not shown), the working end 415 and sleeve 428 may be configured to axially reciprocate together with the endoscope 440 in the shaft assembly 410.

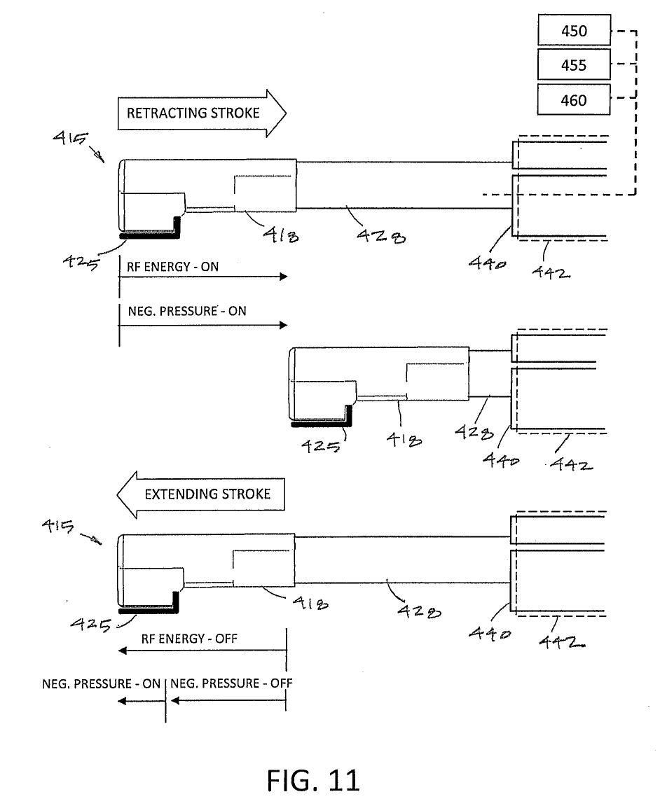

FIG. 11 illustrates a method according to the invention showing retracting and extending strokes of the working end 415 and sleeve 428 wherein a controller 450 activates and de-activates a negative pressure source 455 and causes delivery of RF current from an RF source 460 to the moveable electrode 425 in different portions of the retracting and extending strokes.

The methods of the present invention can employ any tissue resecting device having a moveable working end such as working end 415 and moveable sleeve 428 described previously, extending along a longitudinal axis to a distal housing 418 and having a window, such as window 420 in communication with a remote negative pressure source 455, a moveable electrode 425 configured to move relative to the window 420 and at least one motor 405 adapted to move the electrode across the window 420 and optionally to reciprocate or otherwise move the working end 415 in an axial stroke. The motor drive 405 can be adapted to rotationally oscillate the electrode at any of the rates set forth previously herein, often being greater than 1 CPS (cycles per second) relative to the window. Optionally, the motor can be used to axially reciprocate the sleeve 428 and working end 415 at least once every 2 seconds or at least once per second relative to the handle.

In another variation, the tissue resecting device is coupled to a controller 450 that is configured to operate (1) the RF source 460 coupled to the electrode, (2) the negative pressure source 455, and (3) the at least one motor 405 for moving the electrode 425 and optionally for reciprocating the working end 415 within the shaft assembly 410. Further, the controller may be adapted to control the at least one motor drive to stop movement of the electrode 425 in a selected position relative to the window 420. More in particular, the controller can be adapted to selectively stop the electrode 425 in the center of the window 420 or at an edge of the window.

In still further variations, the controller 450 is adapted to control the at least one motor drive 405 to provide a single movement or cycle of the electrode 425 back and forth across the window 420. In yet another variation, the controller 450 is adapted to control the at least one motor to stop movement of the working end 415 and sleeve 428 in a selected axial position relative to the shaft assembly 410.

Referring again to FIG. 11, the controller 450 can be adapted to control the at least one motor drive 405 to provide a single movement of the shaft assembly in a retracting and extending stroke. In another embodiment, the controller 450 is configured to operate the RF source 460, the negative pressure source 455 and the at least one motor drive 405 contemporaneously. For example, the controller 450 can be adapted to modulate the negative pressure source 455 in response to movement of the working end, or activate or de-activate the RF source in response to movement of the working end, or modulate the negative pressure source in response to movement of the electrode 425 relative to the window, or activate or de-activate the RF source in response to movement of the electrode 425 relative to the window 420 in ceramic body 418. Further, the RF source 460 can be configured to deliver a cutting current waveform or a coagulation waveform to the electrode.

Referring to FIG. 11, a method of resecting tissue according to the present invention comprises providing an elongate shaft assembly, such as assembly 410, having a longitudinal axis and including a reciprocating sleeve 428 carrying a working end 415 comprising a distal housing 418 having an electrode 425 proximate a window 420 in the housing. The sleeve 428 and working end 415 are moveable relative to a stationary portion of the handle 402 with a retracting stroke and an extending stroke. The working end 415 is positioned against a targeted tissue site, and a negative pressure source communicating with the window 420 in the working end 415 is activated. An RF source is activated to deliver RF current to the electrode 425 as the motor drive moves the electrode across the window, and the working end 415 is moved in a retracting stroke to thereby resect tissue while the negative pressure source remains activated to draw tissue into contact with the window 420. The method may further comprise de-activating the negative pressure source, the motor drive and typically also the RF source at the proximal end of the retracting stroke, typically via the controller. Subsequently, the method may comprise commencing the extending stroke with the negative pressure source de-activated and with the RF source de-activated. As can be seen in FIG. 11, the controller activates the negative pressure source during a terminal portion of the extending stroke to again draw tissue into contact with the window 420 to prepare for the following retracting stroke which then again resects tissue with the energized, oscillating electrode 425.

As can be understood from the steps of the method described above, variations of the timing of activation and de-activation of the negative pressure source and RF current delivery are possible. In another variation, the electrode can be energized and oscillated to resect tissue in both the retracting stroke and the extending stroke with the negative pressure source continuously activated.

In another variation, the electrode can be stopped in a selected position in the window, and a coagulation current can be delivered to the electrode for coagulating tissue. Alternatively, the cutting current waveform can be delivered to the stationary electrode for ablating tissue.

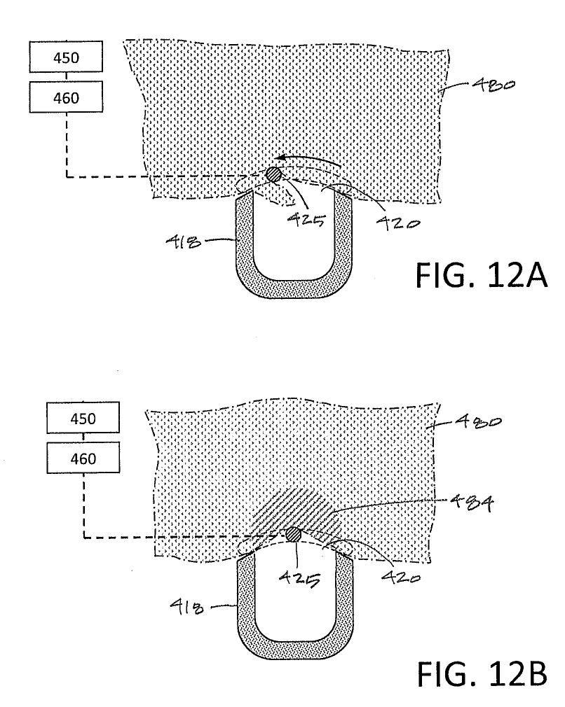

FIGS. 12A-12B illustrate another aspect of the present invention wherein the controller 450 and RF source 460 can be adapted to deliver an RF current with a cutting waveform to the electrode 425 or an RF current with a coagulation waveform to the electrode in various modes of electrode movement or when the electrode is stationary relative to the window. FIGS. 12A-12B are sectional views of the working end of FIG. 9A or FIG. 11 interfacing or engaging with tissue 480.

In general, a method of treating prostate tissue comprises providing a treatment device with a shaft extending along a longitudinal axis to a distal portion having a window 420 in ceramic body 418 communicating with a negative pressure source and a motor driven electrode 425 adapted to move relative to the window, positioning the window in an interface with targeted tissue 480, operating in a first mode with a cutting waveform delivered to the electrode while activating the motor to move the electrode to resect tissue 480 (FIG. 12A) and thereafter operating in a second mode with a coagulation waveform delivered to the electrode 425 after de-activating the motor to stop the electrode 425 in a selected stationary position to coagulate tissue indicated at 484 (FIG. 12B). Further, the positioning step can be preceded by the step of introducing the shaft in a trans-urethral approach into a patient's prostate. The first mode includes sweeping the electrode 425 across the window 420 to resect tissue interfacing the window as shown in FIG. 12A. The electrode 425 can be adapted to sweep across the window from side to side, or in another variation can move distally and proximally in the window 430.

In the first mode, the electrode 425 can move at a rate of greater than 1 CPS relative to the window 430. Further, operating in the first mode includes activating the aspiration source within a first negative pressure range to draw tissue against or into the window and to aspirate fluid and resected tissue through the window. Operating in the second mode includes activating the aspiration source within a second negative pressure range to aspirate fluid through the channel in the shaft. When operating in the first and second modes, a controller is utilized to activate and de-activate the motor, the RF source and the negative pressure source in a selected manner.

In another method, the controller can operate the motor and RF source in a third mode to delivering a coagulation waveform while activating the motor to move the electrode at less than 100 CPS.

In another method, the controller can operate the motor and RF source in a fourth mode delivering a cutting waveform after de-activating the motor to stop the electrode in a selected stationary position.

When the device is operated in a mode with a stationary electrode, the selected stationary position of the electrode is substantially centered in the window. Such a centered position allows for aspiration of fluid around both sides of the electrode through the window which cools the electrode in the coagulation mode and remove bubbles when the cutting current is used to ablated tissue.

In general, a tissue resecting device comprises an elongated shaft extending along a longitudinal axis to a distal portion having a window communicating with an aspiration source, a wire-like electrode configured to move relative to the window, an RF source configured to deliver RF current in a cutting waveform and a coagulation waveform to the electrode, a motor configured to move the electrode, and a controller configured to operate the motor and RF source in a first mode delivering a cutting waveform while activating the motor to move the electrode, and in a second mode delivering a coagulation waveform after de-activating the motor to stop the electrode in a selected stationary position. In this variation, the electrode has a surface area smaller than the window area to permit fluid aspiration around the electrode and through the window in the first and second operating modes.

When operating in the first mode, the controller can activate the aspiration source within a first negative pressure range. When operating in the second mode, the controller can activate the aspiration source within a second negative pressure range.

When operating in a third mode, the controller can be configured to operate the motor drive and RF source to deliver a coagulation waveform while activating the motor to move the electrode at less than 50 CPS.

When operating in a fourth mode, the controller can be configured to operate the motor and RF source to deliver a cutting waveform after de-activating the motor to stop the electrode in a selected stationary position, for example in the center of the window.

As can be seen in FIGS. 9A, 9B and 11, the distal portion of the sleeve 428 includes a dielectric body or housing 418 having the window 420 therein. Typically, the housing is a ceramic material which can be selected from the group consisting of yttria-stabilized zirconia, magnesia-stabilized zirconia, ceria-stabilized zirconia, zirconia toughened alumina and silicon nitride.

The motor drives shown in FIGS. 8, 10A and 10B can be disposable or detachable and thus re-usable.

As can be understood from the steps of the method described above, variations of the timing of activation and de-activation of the negative pressure source and RF current delivery are possible. In another variation, the electrode can be energized to resect tissue in both the retracting stroke and the extending stroke with the negative pressure source continuously activated.

In another variation, the electrode can be stopped in a selected position in the window, and a coagulation current can be delivered to the electrode for coagulating tissue. Alternatively, the cutting current waveform can be delivered to the stationary electrode for ablating tissue.

What is claimed is:

1. A tissue resecting probe comprising:
an elongated shaft extending along a longitudinal axis to a distal portion having a window communicating with an aspiration source;
a wire-like electrode configured to move relative to the window;
an RF source configured to deliver RF current in a cutting waveform and a coagulation waveform to the electrode;
a motor configured to move the electrode; and
a controller configured to operate the motor and RF source in a first mode delivering a cutting waveform while activating the motor to move the electrode, and in a second mode delivering a coagulation waveform after de-activating the motor to stop the electrode in a selected stationary position.

2. The tissue resecting probe of claim 1 where the electrode has a surface area smaller than the window area to thereby permit fluid aspiration around the electrode and through the window in the first and second operating modes.

3. The tissue resecting probe of claim 1 where the electrode extends parallel to the longitudinal axis.

4. The tissue resecting probe of claim 1 where in the first mode, the electrode moves at a rate of equal to or greater than 1 CPS relative to the window.

5. The tissue resecting probe of claim 1 where in the first mode, the electrode moves at a rate of greater than 1 CPS relative to the window.

6. The tissue resecting probe of claim 1 wherein the controller in the first mode activates the aspiration source within a first negative pressure range.

7. The tissue resecting probe of claim 1 wherein the controller in the second mode activates the aspiration source within a second negative pressure range.

8. The tissue resecting probe of claim 1 wherein the controller is configured to operate the motor and RF source in a third mode delivering a coagulation waveform while activating the motor to move the electrode at less than 100 CPS.

9. The tissue resecting probe of claim 1 wherein the controller is configured to operate the motor and RF source in a fourth mode delivering a cutting waveform after de-activating the motor to stop the electrode in a selected stationary position.

10. The tissue resecting probe of claim 1 where the electrode in the predetermined stationary position is in the center of the window.

11. The tissue resecting probe of claim 1 where the electrode in the predetermined stationary position is proximate an edge of the window.

12. The tissue resecting probe of claim 1 wherein the distal portion of the shaft includes a dielectric body having the window therein.

13. The tissue resecting probe of claim 12 wherein the dielectric body is a ceramic material.

14. The tissue resecting probe of claim 13 wherein the ceramic material is selected from the group consisting of yttria-stabilized zirconia, magnesia-stabilized zirconia, ceria-stabilized zirconia, zirconia toughened alumina and silicon nitride.

15. A method of treating prostate tissue comprising:
providing a treatment device with a shaft extending along a longitudinal axis to a distal portion having a window communicating with an aspiration source and a motor driven electrode adapted to move relative to the window;
positioning the window in an interface with targeted prostate tissue;
operating in a first mode with a cutting waveform delivered to the electrode while activating the motor to move the electrode to resect tissue; and
operating in a second mode with a coagulation waveform delivered to the electrode after de-activating the motor to stop the electrode in a selected stationary position to coagulate tissue.

16. The method of claim 15 wherein the positioning step is preceded by the step of introducing the shaft trans-urethrally into a patient's prostate.

17. The method of claim 15 wherein the first mode includes sweeping the electrode across the window to resect tissue interfacing the window.

18. The method of claim 15 wherein the electrode sweeps across the window from side to side.

19. The method of claim 15 wherein the electrode sweeps across the window from distally and proximally.

20. The method of claim 15 where in the first mode, the electrode moves at a rate of greater than 1 CPS relative to the window.

21. The method of claim 15 wherein operating in the first mode includes activating the aspiration source within a first negative pressure range to draw tissue against or into the window and aspirate fluid and resected tissue through the window.

22. The method of claim 15 wherein operating in the second mode includes activating aspiration source within a second negative pressure range to aspirate fluid through the channel in the shaft.

23. The method of claim 15 wherein operating in the first and second modes utilizing a controller configured to activate and de-activate the motor, the RF source and the negative pressure source in a predetermined manner.

24. The method of claim 15 wherein the selected stationary position of the electrode allows aspiration of fluid around both sides of the electrode through the window.

25. A tissue resecting device comprising:
a handle;
a shaft assembly movably attached to the handle and having a longitudinal axis;
a housing secured to a distal end of the shaft and having a window configured to be fluidly coupled to a negative pressure source;
an electrode disposed in the housing to move relative to the window; and
at least one motor in the handle adapted to both (1) move the shaft assembly in an axial stroke relative to the handle, and (2) move the electrode across the window.

26. The tissue resecting device of claim 1 wherein the at least one motor is adapted to move the shaft assembly and the electrode contemporaneously.

27. The tissue resecting device of claim 1 wherein the at least one motor is adapted to selectively move either the shaft assembly or the electrode individually.

28. The tissue resecting device of claim 1 wherein the at least one motor is adapted to move the electrode at greater than 1 CPS relative to the window.

29. The tissue resecting device of claim 1 wherein the motor is adapted to reciprocate the shaft assembly at greater once every 2 seconds.

30. A tissue resecting system comprising:
a device according to claim 25; and
a controller configured to operate (1) an RF source configured to be coupled to the electrode, (2) a negative pressure source, and (3) the at least one motor for moving the electrode and the shaft assembly.
